(19) United States
(12) Patent Application Publication
(10) Pub. No.: US 2012/0220910 A1
(43) Pub. Date: Aug. 30, 2012

(54) KNEE SUPPORT DEVICE HAVING ADJUSTABLE OPENINGS AT OPPOSING ENDS

(75) Inventors: Eric Lee Gaylord, Matthews, NC (US); Robert Scott Gaylord, Matthews, NC (US)

(73) Assignee: Medical Specialties, Inc., Charlotte, NC (US)

(21) Appl. No.: 13/036,433

(22) Filed: Feb. 28, 2011

(51) Int. Cl. A61F 5/00

(52) U.S. Cl. 602/16; 602/26

(57) ABSTRACT

The knee support relates to a device for stabilizing movement of the patella and providing lateral support to a knee joint, and in particular, to a knee support device having independently adjustable openings at opposing ends thereof, wherein the adjustable openings accommodate various sized legs and permit a user to independently adjust the compression acting on the proximal and distal sections of the knee joint.

### BACKGROUND OF THE INVENTION

The invention relates to a device for stabilizing movement of the patella and providing lateral support to a knee joint. In particular, the invention relates to a knee support device having independently adjustable openings at opposing ends thereof, wherein the adjustable openings accommodate various sized legs and permit a user to independently adjust the compression acting on proximal and distal zones of the knee.

The knee is one of the most overworked joints in the body. An individual uses the knee joint over one million times per year. As a result of such use, the knee is one of the most injured joints in the body. The knee is more vulnerable to injury because it is one of the most mobile and flexible joints. The more mobile a joint, the less stable the joint; thus, the more vulnerable it is to injury.

The knee is kept in alignment by ligaments and tendons. The ligaments connect the bones and provide stability to joints. There are four main ligaments in the knee joint—anterior cruciate ligament (ACL), the posterior cruciate ligament (PCL), the medial collateral ligament (MCL), and lateral collateral ligament (LCL).

The knee also includes the patellar tendon and the quadriceps tendon. These tendons are connected to the patella. The patellar tendon—located below the patella—is a ropelike structure that connects the bottom of the patella to the top of the tibia. The quadriceps tendon is located above the patella.

The patella slides up and down in a groove in the femur that is referred to as the femoral trochlea or trochlear groove as the knee bends and straightens. Movement of the patella outside of the trochlear groove can result in a subluxation when the patella moves partially out of and back into the groove, or a total patellar dislocation when the patella moves completely out of the groove and remains so.

Patellar subluxations are referred to as mild dislocations. Typically, the patella dislocates or subluxes laterally. One or more patellar subluxations or dislocations may cause the knee to feel unstable. This type of problem is referred to as patellar instability. This instability occurs because the muscles and ligaments are unable to keep the patella in the trochlear groove. Dislocation is the most severe form of patellar instability. Thus there is a need for a support device that is capable of stabilizing patellar movement and restricting movement of the patella out of the trochlear groove, while providing enhanced adjustability.

### SUMMARY OF THE INVENTION

The invention 10 comprises in one embodiment a flexible sleeve 11 defining at least one opening 12, first and second openings 13, 14 defined by opposing ends of the sleeve, vertical slits 15, 16 defined by proximal and distal sections of the sleeve, first and second tensioning flaps 20, 21 extending laterally from opposing ends of the sleeve, stiffening members 22, 23 having an upper part 22a, 23a and a lower part 22b, 23b connected at a pivot point 24, a padded buttress 25 removably attachable to a side of the opening 12, and corresponding fasteners 30, 32 positioned on the sleeve and tensioning straps for securing the sleeve to a knee. The tensioning flaps 20, 21 extend across a minimal portion of the front of the sleeve 11 proximal and distal of the knee. Advantageously, the first and second openings 13, 14 are independently adjustable to accommodate various sized legs and to permit a user to adjust the compression acting on the knee joint. The invention also includes an adjustable proximal zone 33, a fixed (or non-adjustable) intermediate or central zone 34, and an adjustable distal zone 35. The first and second openings 13, 14 are located in the proximal and distal zones 33, 35 (with respect to the intermediate or central zone 34 of the sleeve 11 surrounding the knee joint) and are independently adjustable to accommodate various sized legs in the zones. The positioning of the zones 33, 35 (i.e., proximal and distal relative to the central zone 34) fundamentally relates to the uniqueness of the invention in that most importantly and foremost, appropriate compression can be provided via proper device sizing specifically for the central knee joint zone. Subsequently the proximal and distal zones 33, 35 can be independently adjusted to accommodate the typically widely varied human anatomies (for a given central zone size) above and below the knee joint.

In another embodiment, the invention may include at least one tensioning strap (e.g., first and second tensioning straps 37, 38) that extends circumferentially around the sleeve 11 proximal and distal of the knee. The tensioning straps 37, 38 and tensioning flaps 21, 22 are secured to the sleeve 11 at one end.

Advantageously, the invention 10 bears weight during both flexion and extension of the knee. Normal use of the invention 10 also accommodates stress and strain associated with rotation and lateral forces.

### BRIEF DESCRIPTION OF THE DRAWING

The foregoing and other objects and advantages of the invention and the manner in which the same are accomplished will become clearer based on the following detailed description taken in conjunction with the accompanying drawing in which various embodiments of the invention are depicted.

FIG. 1 is a partial cross-sectional side view of one embodiment of the invention;

FIG. 2 is a partial cross-sectional front view of one embodiment of the invention;

FIG. 3 is a rear view of one embodiment of the invention;

FIG. 4 is a partial top plan view of one embodiment of the invention taken along line 4-4 of FIG. 2;

FIG. 5 is a partial top plan view of one embodiment of the invention taken along line 5-5 of FIG. 2;

FIG. 6 is a partial enlarged side view of a portion of one embodiment of the present invention;

FIG. 7 is a partial enlarged front view of a portion of one embodiment of the present invention taken along line 7-7 of FIG. 6;

FIG. 8 is a partial enlarged cross-sectional front view of one embodiment of the present invention;

FIG. 9 is a top plan view of a support member of one embodiment of the invention; and

FIG. 10 is a partial cross-sectional front view of another embodiment of the invention.

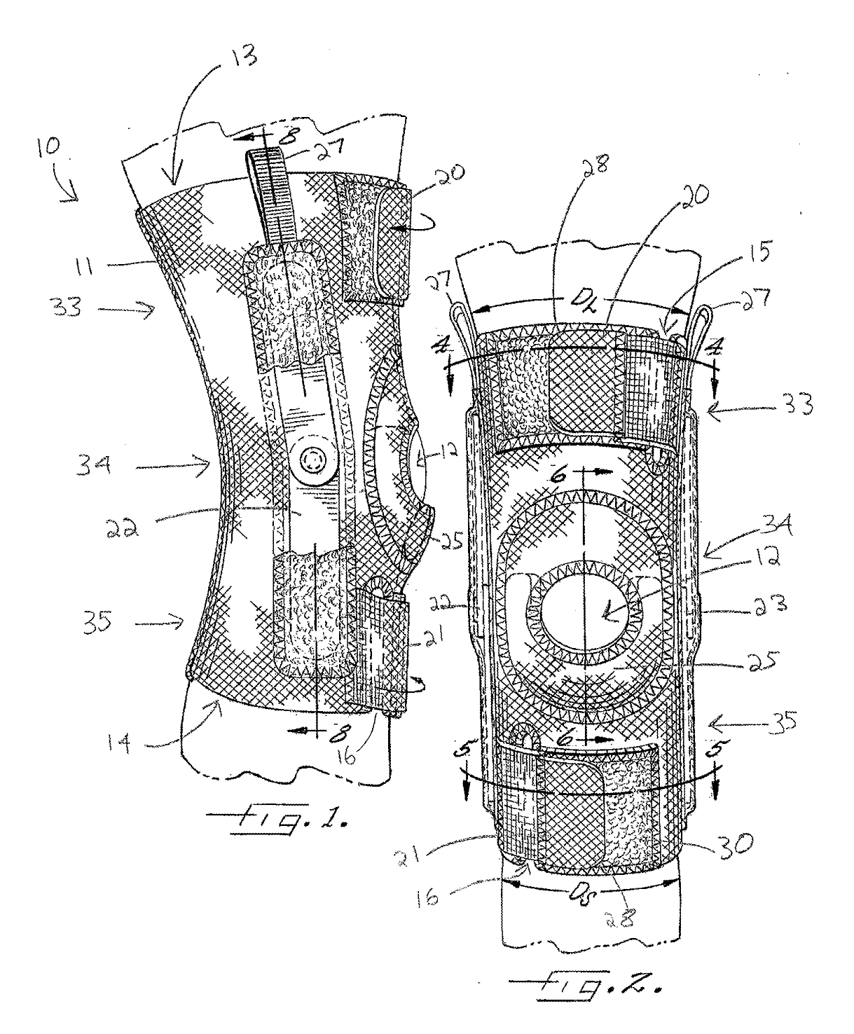

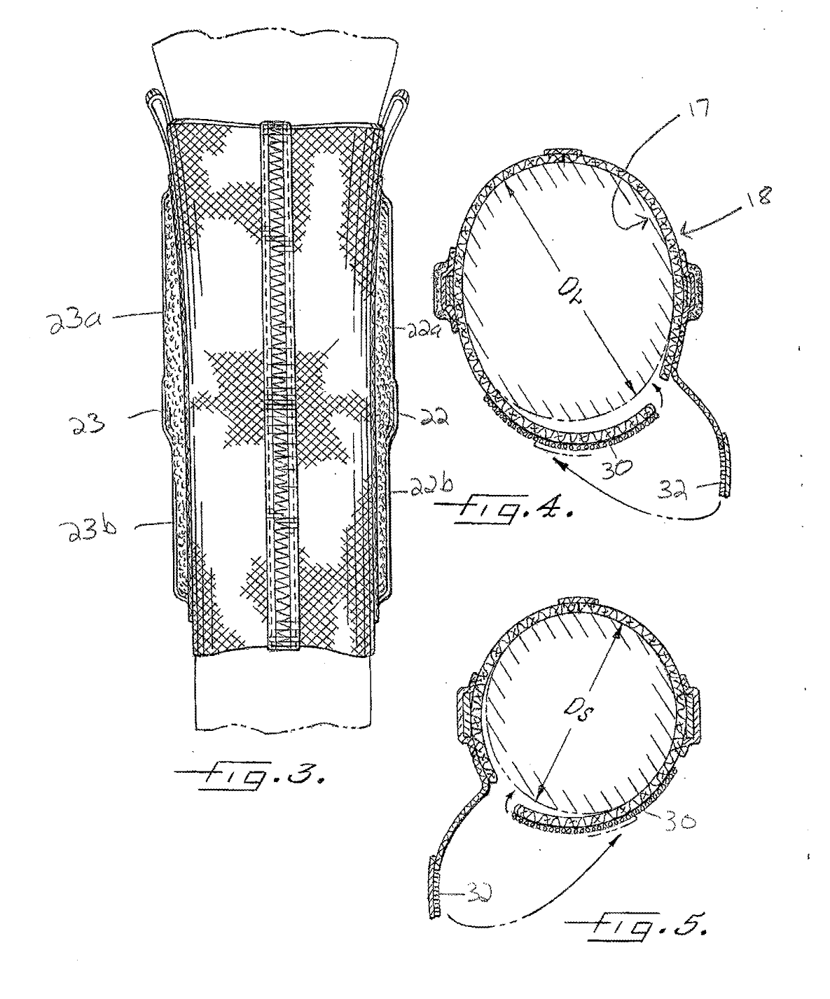

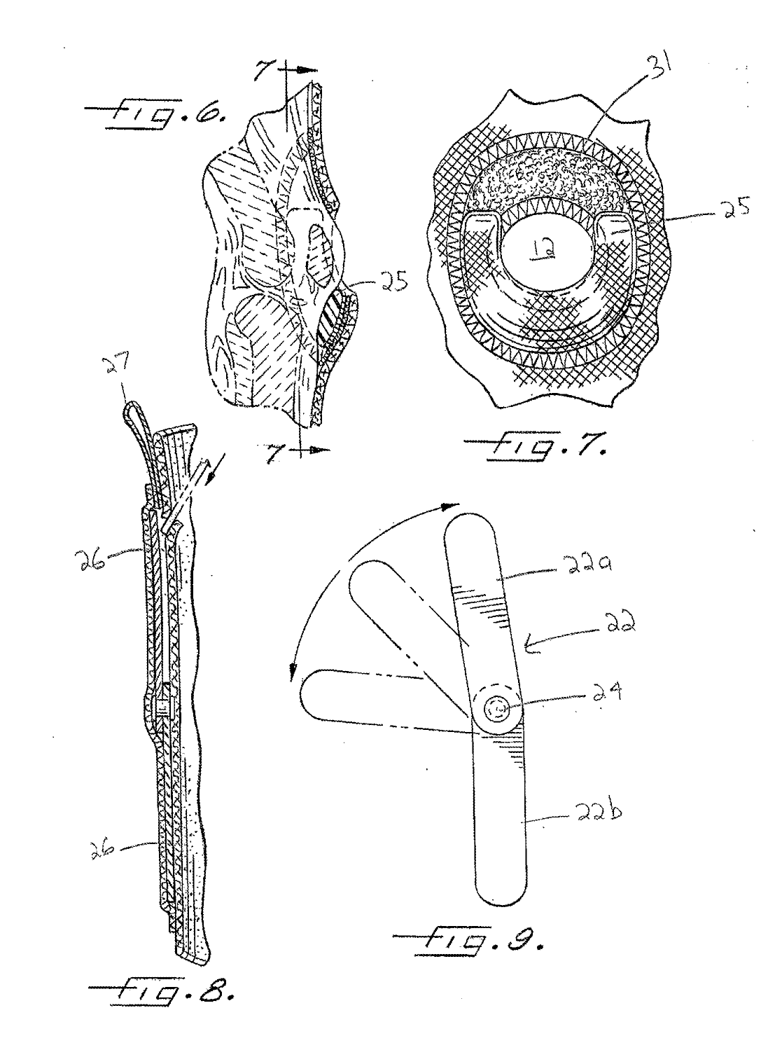

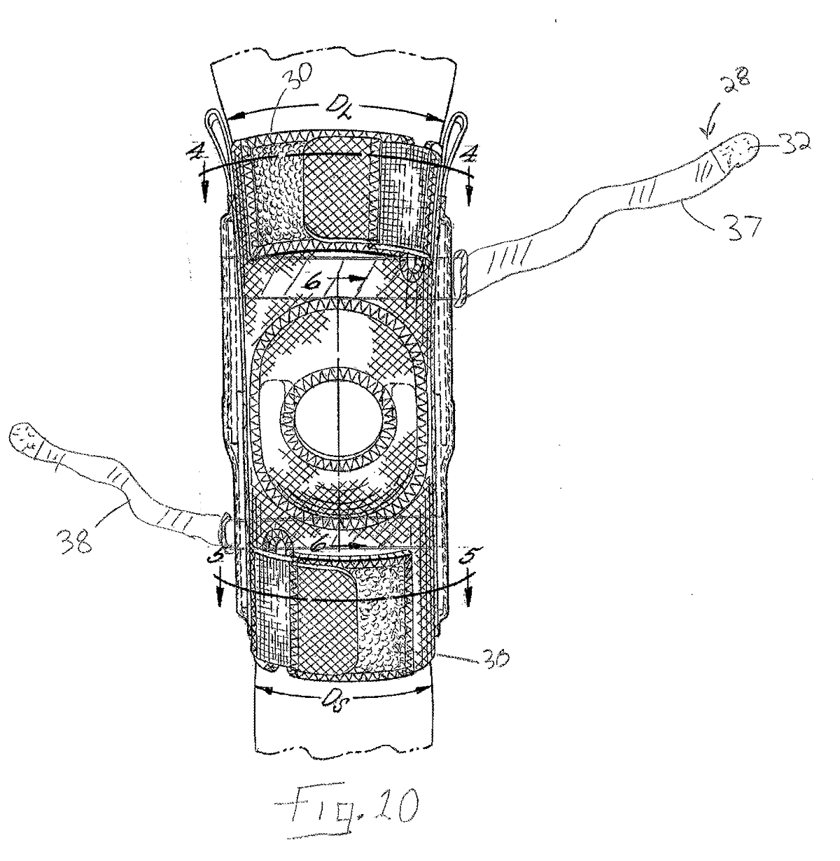

### DETAILED DESCRIPTION OF THE INVENTION

The present invention will now be described more fully hereinafter with reference to the accompanying drawings, in which a preferred embodiment of the invention is shown. This invention may, however, be embodied in many different forms and should not be construed as limited to the embodiments set forth herein. Rather, these embodiments are provided so that this disclosure will be thorough and complete, and will fully convey the scope of the invention to those skilled in the art. Like numbers refer to like elements throughout.

The invention 10 comprises in one embodiment a flexible sleeve 11 defining at least one opening 12 between opposing ends of the sleeve, first and second openings 13, 14 defined by opposing ends of the sleeve, first and second vertical slits 15, 16 defined by proximal and distal sections of the sleeve, first and second tensioning flaps 20, 21 extending laterally from opposing ends of the sleeve, first and second stiffening members 22, 23 having an upper part and a lower part connected at a pivot point 24, a padded buttress 25 removably attachable to at least one side of the patellar opening 12, and corresponding fasteners 30, 32 positioned on the sleeve and tensioning straps for securing the sleeve to a knee. The flexible sleeve may be formed from an elastomeric material. In one embodiment, the first and second tensioning flaps 20, 21 include free ends 28 that extend across and are releasably secured to a front portion of the sleeve. In another embodiment, the invention 10 includes tensioning straps 37, 38 that extend circumferentially around the sleeve 11 proximal and distal of the knee, and are releasably secured to a side portion of the sleeve. In both embodiments, the first and second tensioning flaps 20, 21 and first and second tensioning straps 37, 38 may be formed at least in part from an elastic material.

The flexible sleeve 11 has an interior and exterior surface 17, 18. The exterior surface 17 includes fasteners 30 for removably attaching at least a portion of the first and second tensioning flaps 20, 21 (and optionally the first and second tensioning straps 37, 38) to the sleeve. The interior surface 18 also includes fasteners 30 for removably attaching at least a portion of the padded buttress 25 to the sleeve. The fasteners 30 may include loops for engaging hooks secured to other portions of the device.

The opening 12 positioned between opposing ends of the sleeve 11 is configured to receive the patella of the knee when the device is placed on the leg and knee joint.

The first and second openings 13, 14 at opposing ends of the sleeve 11 are configured to receive portions of the leg and knee joint. The openings 13, 14 are adjustable to accommodate various sized legs and permit a user to adjust the compression acting on the knee joint (e.g., diameter L or D_{L} and diameter S or D_{S} may vary as depicted in FIG. 2). In novel fashion, the openings 13, 14 are rendered adjustable by first and second vertical slits 15, 16 defined by proximal and distal zones or sections 33, 35 of the sleeve. It will be understood that the device may include one or more vertical slits 15, 16. The vertical slits 15, 16 extend the length of at least a portion of the flexible sleeve 11 and are vertically offset from one another. Stated differently, the slits 15, 16 are offset with respect to one another as defined by a vertical axis extending the length of the sleeve.

The first and second tensioning flaps 21, 21 extend laterally from the proximal and distal ends of the flexible sleeve 11. The first and second tensioning flaps 20, 21 are secured to the flexible sleeve 11 substantially adjacent to the first and second slits 15, 16. In other words, the first and second tensioning flaps 20, 21 are secured substantially adjacent to opposing edges of the first and second slits 15, 16, respectively. Accordingly, the tensioning flaps 20, 21 are extended against the sleeve in opposing directions when fitted to the leg and knee, thereby enhancing a secure and adjustable fit. The first and second tensioning flaps 20, 21 have fasteners 32 at free ends for removably attaching at least a portion of the tensioning flaps to the flexible sleeve 11. Likewise, the first and second tensioning straps 37, 38 have fasteners 32 at free ends for removably attaching at least a portion of the tensioning straps to the flexible sleeve 11.

The first and second stiffening members 22, 23 have an upper part 22a, 23a and a lower part 22b, 23b that are connected at a pivot point 24. The first and second stiffening members 22, 23 are positioned against opposing sides of the flexible sleeve 11 (i.e., lateral and medial sides of the device). The stiffening members 22, 23 may be formed of semi-rigid plastic or similar material capable of providing lateral support to the knee joint. The stiffening members 22, 23 may be secured against the sleeve 11 in pockets 26 on the sleeve. The pivot point 24 permits the upper and lower parts 22a, 23a, 22b, 23b of the stiffening members 22, 23 to move with the knee when the device is worn.

The padded buttress 25 is removably attachable to at least one side of the patellar opening 12. The padded buttress 25 has fasteners 31 for removably attaching at least a portion of the buttress to at least one side of the patellar opening 12. The padded buttress 25 is substantially arcuate and concave with respect to the patellar opening 12.

The device 10 may also include at least one positioning strap 27 secured to a proximal end of the flexible sleeve 11. The strap or straps 27 provide a point for a user to grasp the device and pull it up into position over the leg and knee joint during application.

In the drawings and specification, there have been disclosed typical embodiments on the invention and, although specific terms have been employed, they have been used in a generic and descriptive sense only and not for purposes of limitation, the scope of the invention being set forth in the following claims.

That which is claimed is:

1. A device for supporting a knee joint comprising:
a flexible sleeve defining at least one opening positioned between opposing ends of said sleeve;
first and second openings defined by opposing ends of said flexible sleeve;
first and second tensioning flaps extending laterally from opposing ends of said flexible sleeve, said first and second tensioning flaps secured to said flexible sleeve;
first and second stiffening members having an upper part and a lower part connected at a pivot point, the first and second stiffening members positioned against opposing sides of the flexible sleeve; and
a padded buttress removably attachable to at least one side of said at least one opening;
wherein the first and second openings are independently adjustable to accommodate various sized legs and to permit a user to adjust the compression acting on the knee joint.

2. A device according to claim 1 wherein said first and second openings are rendered adjustable by first and second vertical slits defined by proximal and distal sections of the sleeve.

3. A device according to claim 2 wherein said vertical slits extend the length of at least a portion of said flexible sleeve and are vertically offset from one another.

4. A device according to claim 2 wherein said first and second tensioning flaps are secured substantially adjacent to opposing edges of said first and second slits, respectively.

5. A device according to claim 1 wherein free ends of said first and second tensioning flaps extend across and are releasably secured to a front portion of said sleeve.

6. A device according to claim 1 wherein said first and second tensioning flaps have fasteners for removably attaching at least a portion of said first and second tensioning flaps to said flexible sleeve.

7. A device according to claim 1 wherein said padded buttress has fasteners for removably attaching at least a portion of said buttress to at least one side of said at least one opening.

8. A device according to claim 1, wherein said flexible sleeve has an interior and exterior surface, said exterior surface having fasteners for removably attaching at least a portion of said first and second tensioning flaps thereto, said interior surface having fasteners for removably attaching at least a portion of said padded buttress thereto.

9. An apparatus according to claim 1, wherein said flexible sleeve is formed from an elastomeric material.

10. An apparatus according to claim 1, wherein said first and second tensioning flaps are formed at least in part from an elastic material.

11. An apparatus according to claim 1, wherein said padded buttress is substantially arcuate and concave with respect to said at least one opening.

12. A device according to claim 1 further comprising at least one positioning strap secured to a proximal end of said flexible sleeve for applying said flexible sleeve to a knee joint.

13. A device according to claim 1 further comprising at least one tensioning strap having a free end that extends circumferentially around said sleeve and is releasably secured to a side portion of said sleeve.

14. A device for supporting a knee joint comprising:
a flexible sleeve defining at least one opening positioned between opposing ends of the sleeve, said sleeve further defining an adjustable proximal zone, a fixed intermediate zone, and an adjustable distal zone;
first and second openings defined by opposing ends of said flexible sleeve, said first and second openings rendered adjustable by first and second vertical slits defined by proximal and distal sections of said sleeve, said first and second vertical slits extending the length of at least a portion of said flexible sleeve and vertically offset from one another;
first and second tensioning flaps extending laterally from opposing ends of said flexible sleeve, said first and second tensioning flaps secured to said flexible sleeve substantially adjacent to said first and second slits;
first and second stiffening members having an upper part and a lower part connected at a pivot point, said first and second stiffening members positioned against opposing sides of said flexible sleeve; and
a padded buttress removably attachable to at least one side of said at least one opening;
wherein said first and second openings are independently adjustable about said proximal and distal zones to accommodate various sized legs and to permit a user to adjust the compression acting on the knee joint.

15. A device according to claim 14 wherein free ends of said first and second tensioning flaps extend across and are releasably secured to a front portion of said sleeve.

16. A device according to claim 14 further comprising first and second tensioning straps having free ends that extend circumferentially around said sleeve and are releasably secured to a side portion of said sleeve.

17. A device for supporting a knee joint comprising:
a flexible sleeve defining at least one patellar opening, said at least one opening positioned between opposing ends of the sleeve, said sleeve further defining an adjustable proximal zone, a fixed intermediate zone, and an adjustable distal zone;
first and second openings defined by opposing ends of the flexible sleeve, said first and second openings rendered adjustable by first and second vertical slits defined by proximal and distal sections of said sleeve, said vertical slits extending the length of at least a portion of said flexible sleeve and vertically offset from one another;
first and second tensioning flaps extending laterally from opposing ends of said flexible sleeve, said first and second tensioning flaps secured to said flexible sleeve substantially adjacent to said first and second slits;
first and second stiffening members having an upper part and a lower part connected at a pivot point, said first and second stiffening members positioned against opposing sides of said flexible sleeve for supporting the knee joint; and
a padded buttress removably attachable to an interior portion of said sleeve and to at least one side of said at least one opening, said padded buttress for securing the patella of the knee joint.

18. A device according to claim 17 wherein free ends of said first and second tensioning flaps extend across and are releasably secured to a front portion of said sleeve.

19. A device according to claim 17 wherein said first and second openings are independently adjustable about said proximal and distal zones to accommodate various sized legs and to permit a user to adjust the compression acting on the knee joint.

20. A device according to claim 17 further comprising first and second tensioning straps that extend circumferentially around said sleeve and are releasably secured to a side portion of said sleeve.
